(19) United States
(12) Patent Application Publication
(10) Pub. No.: US 2015/0209507 A1
(43) Pub. Date: Jul. 30, 2015

(54) SYRINGE

(71) Applicant: Coeur, Inc., Lebanon, TN (US)

(72) Inventor: J. Michael Cude, College Grove, TN (US)

(21) Appl. No.: 14/417,413

(22) PCT Filed: Aug. 13, 2013
(86) PCT No.: PCT/US2013/054676

§ 371 Date: Jan. 26, 2015

Related U.S. Application Data
(60) Provisional application No. 61/683,625, filed on Aug. 15, 2012.

(51) Int. Cl. A61M 5/145; A61M 5/31

(52) U.S. Cl. CPC A61M 5/14546; A61M 5/3129; A61M 2005/14553

(57) ABSTRACT

A syringe for a power injector includes a barrel having a distal end and a proximal end. The barrel defines an interior volume therebetween the distal and proximal ends. A circumferential flange extends about the proximal end. A delivery tip extends from the distal end. The barrel includes a thick wall section adjacent the distal end and a thin wall section adjacent the proximate end.

### CROSS-REFERENCE TO RELATED APPLICATION

This application claims priority to and the benefit of U.S. Provisional Application No. 61/683,625, filed Aug. 15, 2012.

### BACKGROUND

This invention relates in general to medical injection systems.

One type of medical injection system includes a syringe and a power-driven injector. For example, for intravenous delivery the syringe may be filled with fluid to delivery and the syringe may be connected to a patient by appropriate tubing and a needle. Typically, in such an arrangement, the injector will include a plunger which engages a plunger jacket inside the syringe to push fluid from the syringe to a patient.

Such syringes are typically made of plastic. Due to the high pressures generated during the delivery process such syringes may include an outer pressure jacket. Alternatively, a syringe with a relatively thicker outer wall may be used and then not include an outer pressure jacket.

### SUMMARY

This invention relates more specifically to syringes for medical injection systems.

In one embodiment a syringe for a power injector includes a barrel having a distal end and a proximal end. The barrel defines an interior volume therebetween the distal and proximal ends. A circumferential flange extends about the proximal end. A delivery tip extends from the distal end. The barrel includes a thick wall section adjacent the distal end and a thin wall section adjacent the proximate end.

The syringe may defines a common inner surface for engagement of a plunger jacket shared by the thick wall section and the thin wall section.

The syringe may be formed from thermo-plastic, such as polypropylene.

The thick wall section may have a greater pressure capability than the thin wall section. The thick wall section may have a pressure capability of at least 460 psi. The thin wall section may have a pressure capability of at least 375 psi.

Various aspects will become apparent to those skilled in the art from the following detailed description and the accompanying drawings.

### BRIEF DESCRIPTION OF THE DRAWINGS

FIG. 1 is a perspective view of a injection system as shown in exploded view.

FIG. 2 is an enlarged perspective view of the syringe of FIG. 1.

FIG. 3 is a cross-sectional view of the syringe of FIG. 2.

### DETAILED DESCRIPTION

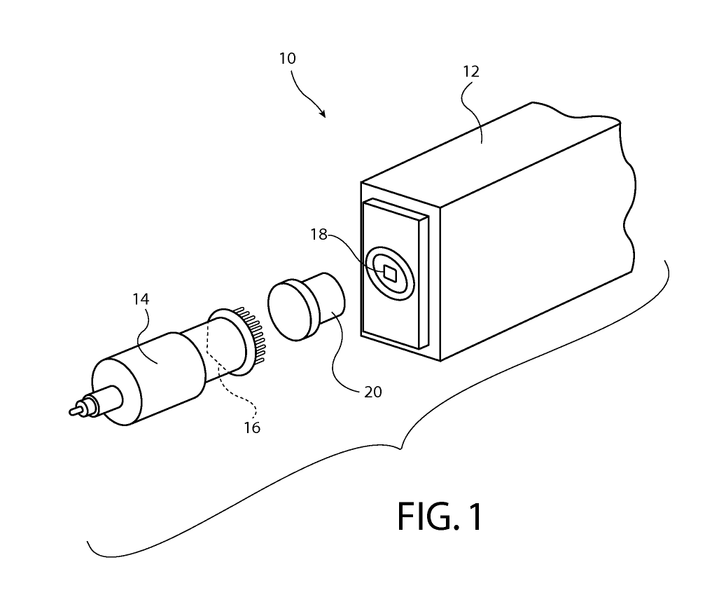

Referring now to the drawings, there is illustrated in FIG. 1 an injection system 10. The system 10 includes a power driven injector 12 and a syringe 14. A plunger jacket 16 is optionally disposed within the syringe 14. The injector 12 includes a ram 18 that drives a plunger 20 that engages the plunger jacket 16 and thus may push fluid from the syringe 14.

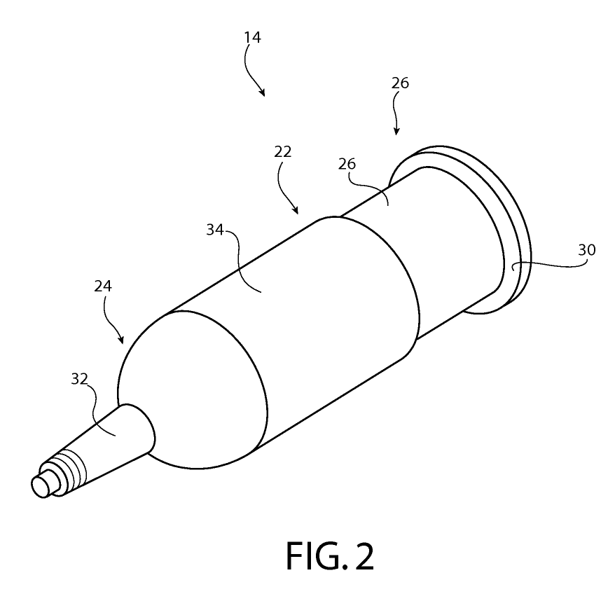

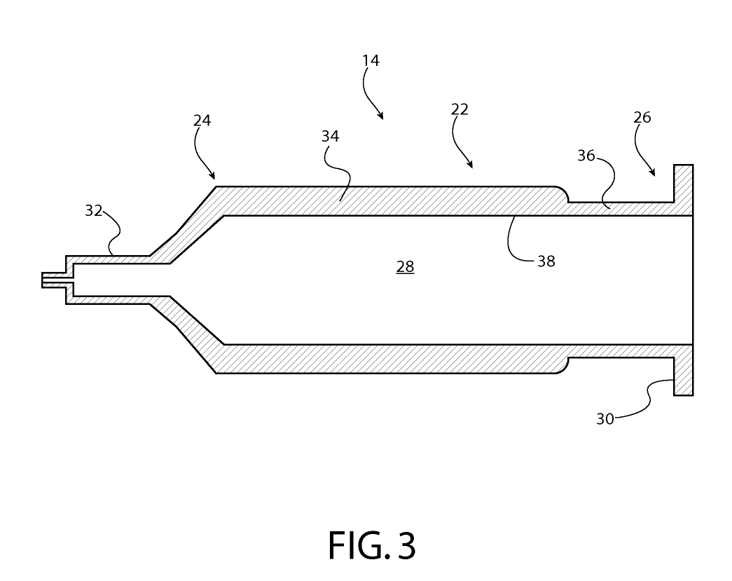

As best shown in FIGS. 2 and 3, the syringe 14 includes a barrel 22. The barrel 22 has a distal end 24 and a proximal end 26. The barrel 22 defines an interior volume 28 therebetween the distal end 24 and the proximal end 26. A circumferential flange 30 extends about the proximal end 26. A delivery tip 32 extends from the distal end 24.

The barrel 22 includes a thick wall section 34 adjacent the distal end 24. The barrel 22 includes a thin wall section 36 adjacent the proximate end 26. The thick wall section 34 and the thin wall section 36 are generally thick and thin respectively relative to one another. The thick wall section 34 and the thin wall section 36 share a common inner surface 38 for engagement of the plunger jacket 16.

In one embodiment the syringe 14 is formed from an olefin or thermo-plastic, such as Polypropylene.

The thick wall section 34 has a greater pressure capability than the thin wall section 36. For example the thick wall section 34 may be rated to 460 psi while the thin wall section 36 is rated for 375 psi. Thus in at least one embodiment the thick wall section may have a pressure capability of at least 460 psi, and in at least one embodiment the thin wall section may have a pressure capability of at least 375 psi.

It has been discovered that the pressure of a fluid in the interior volume 28 increases as the plunger 20 and plunger jacket 16 move along the axial length of the barrel 22 from the proximal end 26 toward the distal end 24. In operation the syringe 14 with a syringe jacket, i.e. a jacketless syringe, may withstand greater pressure in use than an otherwise thinner unjacketed syringe, while having a relatively thinner wall section adjacent the proximate end.

In one operation, the syringe 14 is filled with a fluid for an injection operation. The syringe 14 with plunger jacket 16, the plunger 20, and without a syringe jacket, is loaded on the power driven injector 12 with the ram 18 engaging the plunger 20 and the plunger jacket 16 to push fluid from the syringe 14 with the pressure in the syringe increasing as the ram 18 moves along the syringe 14.

While principles and modes of operation have been explained and illustrated with regard to particular embodiments, it must be understood, however, that this may be practiced otherwise than as specifically explained and illustrated without departing from its spirit or scope.

What is claimed is:

1. A syringe for a medical power injector comprising:
a barrel having a distal end and a proximal end and a defining an interior volume therebetween,
a circumferential flange extending about the proximal end, and
a delivery tip extending from the distal end,
wherein the barrel includes a thick wall section adjacent the distal end and a thin wall section adjacent the proximate end.

2. The syringe of claim 1 wherein the barrel defines a common inner surface for engagement of a plunger jacket shared by the thick wall section and the thin wall section.

3. The syringe of claim 1 wherein the syringe is formed from thermo-plastic.

4. The syringe of claim 3 wherein the thermo-plastic is polypropylene.

5. The syringe of claim 1 wherein the thick wall section has a greater pressure capability than the thin wall section.

6. The syringe of claim 5 wherein the thick wall section has a pressure capability of at least 460 psi.

7. The syringe of claim 5 wherein the while the thin wall section has a pressure capability of at least 375 psi.

8. A method for operating a medical injector system comprising:
providing a power injector with a ram,
providing a syringe having:
a barrel having a distal end and a proximal end and a defining an interior volume therebetween,
a circumferential flange extending about the proximal end, and
a delivery tip extending from the distal end,
wherein the barrel includes a thick wall section adjacent the distal end and a thin wall section adjacent the proximate end, and
loading the syringe on the power injector.

9. The method of claim 8 further comprising, prior to loading the syringe on the power injector, filling the syringe with a fluid for injection.

10. The method of claim 8 wherein the loading includes loading the syringe on the power injector with a plunger and a plunger jacket engaging the ram

11. The method of claim 10 further comprising:
driving the ram to push fluid from the syringe.

12. The method of claim 11 wherein the driving includes increasing pressure in the syringe as the ram moves along the syringe.

13. A medical system for injecting a fluid comprising:
a syringe for a power injector including:
a barrel having a distal end and a proximal end and a defining an interior volume therebetween,
a circumferential flange extending about the proximal end, and
a delivery tip extending from the distal end,
wherein the barrel includes a thick wall section adjacent the distal end and a thin wall section adjacent the proximate end, and
a power injector with a ram for pushing fluid from the syringe.

14. The system of claim 13 further comprising:
a plunger and a plunger jacket engaging the ram and disposed within the syringe.

15. The system of claim 14 wherein the barrel defines a common inner surface for engagement of a plunger jacket shared by the thick wall section and the thin wall section.

16. The system of claim 14 wherein the syringe is formed from thermo-plastic.

17. The system of claim 16 wherein the thermo-plastic is polypropylene.

18. The system of claim 14 wherein the thick wall section has a greater pressure capability than the thin wall section.

19. The system of claim 18 wherein the thick wall section has a pressure capability of at least 460 psi.

20. The system of claim 18 wherein the while the thin wall section has a pressure capability of at least 375 psi.
